(19) United States
(12) Patent Application Publication
(10) Pub. No.: US 2025/0381230 A1
(43) Pub. Date: Dec. 18, 2025

(54) METHODS AND COMPOSITIONS FOR TREATMENT OF NEURODEGENERATIVE DISORDERS AND REDUCING TAU PROTEIN AGGREGATES

(71) Applicants: SanBio Co. Ltd., Tokyo (JP); Keio University, Tokyo (JP)

(72) Inventors: Sumihiro MAEDA, Tokyo (JP); Hideyuki OKANO, Tokyo (JP); Hirofumi NAKATOMI, Tokyo (JP); Yuta FUKUSHIMA, Tokyo (JP); Shinya HIRATA, Saitama (JP); Toshiki HATANAKA, Tokyo (JP); Sumito KUMAGAI, Tokyo (JP)

(73) Assignees: SanBio Co. Ltd., Tokyo (JP); Keio University, Tokyo (JP)

(21) Appl. No.: 19/222,881

(22) Filed: May 29, 2025

Related U.S. Application Data
(60) Provisional application No. 63/660,097, filed on Jun. 14, 2024.

(51) Int. Cl. A61K 35/28

(52) U.S. Cl. CPC A61K 35/28

(57) ABSTRACT

Disclosed are compositions and methods for treating Alzheimer's disease or for use in treating Alzheimer's disease. Also disclosed are compositions and methods for treating or for use in treating a neurodegenerative disorder characterized by the presence of tau protein aggregates. Furthermore, disclosed are compositions and methods for reducing tau protein aggregates or for use in reducing tau protein aggregates.

### CROSS-REFERENCE TO RELATED APPLICATION

This application claims the benefit of U.S. Provisional Application No. 63/660,097 filed on Jun. 14, 2024, the content of which is incorporated herein by reference in its entirety.

### TECHNICAL FIELD

The present disclosure relates generally to the field of cell therapies, and, more specifically, to cell therapies, compositions, and methods of treatment for neurodegenerative disorders, including Alzheimer's disease. The present disclosure also relates to compositions and methods for reducing tau protein aggregates.

### BACKGROUND

Alzheimer's disease is a type of dementia that is characterized by cognitive decline and memory loss over time [1]. It is estimated that more than 55 million people worldwide have dementia with nearly 10 million new cases every year. Alzheimer's disease is the most common type of dementia and may account for upwards of 60% to 70% of dementia cases [2]. According to one report, more than 11 million family members and other unpaid caregivers provided an estimated 18 billion hours of care to people with Alzheimer's disease and other dementias in the United States alone [1]. Family members and other unpaid caregivers provide, on average, 5 hours of care and supervision for dementia patients per day. Moreover, in the United States, total payments for healthcare, long-term care, and hospice services for dementia patients 65-years and older are estimated to exceed $300 billion [1].

One of the pathological hallmarks of Alzheimer's disease is neurofibrillary tangles (NFTs) made up of hyperphosphorylated tau proteins. NFTs can form when abnormally phosphorylated tau proteins aggregate. These NFTs can impair the ability of neurons to function normally and can cause cell death [3].

To date, many pharmaceutical companies have conducted clinical trials for Alzheimer's therapies. However, most such trials have either failed or showed limited efficacy in patients in the early stages of the disease [4].

Human induced pluripotent stem cells (iPSCs) have been used to model Alzheimer's disease [3] and may serve as a platform for evaluating Alzheimer therapies. Human cells are preferred over murine cells because murine cells have different vulnerabilities and cytotoxic profiles.

Therefore, there is a need for a safe and effective therapies for the treatment of neurodegenerative disorders including Alzheimer's disease. Such a therapy should be capable of being tested on tau protein aggregates formed in human cells and should not adversely affect cells of the nervous system.

### SUMMARY

In some aspects, disclosed is a method for treating Alzheimer's disease. The method can comprise administering a therapeutically effective amount of vandefitemcel to a subject in need thereof.

In some aspects, administering the therapeutically effective amount of vandefitemcel further comprises administering the therapeutically effective amount of vandefitemcel to a brain region of the subject.

In some aspects, administering the therapeutically effective amount of vandefitemcel further comprises administering the therapeutically effective amount of vandefitemcel to at least one of an entorhinal cortex, a corpus callosum, a thalamus, a hypothalamus, an internal capsule, and a cerebral cortex of the subject.

In some aspects, administering the therapeutically effective amount of vandefitemcel further comprises administering the therapeutically effective amount of vandefitemcel to a hippocampus of the subject.

In some aspects, administering the therapeutically effective amount of vandefitemcel further comprises injecting the vandefitemcel at multiple sites within the brain of the subject.

In some aspects, administering the therapeutically effective amount of vandefitemcel further comprises administering the therapeutically effective amount of vandefitemcel to a deposition of tau protein aggregates.

In some aspects, administering the therapeutically effective amount of the vandefitemcel further comprises administering the therapeutically effective amount of the vandefitemcel stereotactically through a burr hole in the skull of a subject.

In some aspects, administering the therapeutically effective amount of vandefitemcel further comprises administering the therapeutically effective amount of vandefitemcel by intracerebral implantation.

In some aspects, administering the therapeutically effective amount of vandefitemcel further comprises administering the therapeutically effective amount of vandefitemcel by parenteral administration.

In some aspects, the therapeutically effective amount of vandefitemcel can be suspended in a sterile isotonic crystalloid solution.

In some aspects, the therapeutically effective amount of vandefitemcel can be between about 1.0 million cells and 10.0 million cells (e.g., about 1.0 million cells, 1.5 million cells, 2.0 million cells, 2.5 million cells, 3.0 million cells, 3.5 million cells, 4.0 million cells, 4.5 million cells, 5.0 million cells, 5.5 million cells, 6.0 million cells, 6.5 million cells, 7.0 million cells, 7.5 million cells, 8.0 million cells, 8.5 million cells, 9.0 million cells, 9.5 million cells, 10.0 million cells, and amounts therebetween).

In some aspects, the vandefitemcel can be made by a method comprising: providing a culture of mesenchymal stem cells; contacting the culture of mesenchymal stem cells with a polynucleotide encoding a Notch intracellular domain (NICD), wherein the polynucleotide does not encode a full-length Notch protein, selecting cells that include the polynucleotide; and further culturing the selected cells in the absence of selection for the polynucleotide.

In some aspects, the mesenchymal stem cells can be human bone marrow-derived cells.

In some aspects, disclosed is a method of treating a neurodegenerative disorder characterized by the presence of tau protein aggregates. The method can comprise administering a therapeutically effective amount of vandefitemcel to a subject in need thereof.

In some aspects, administering the therapeutically effective amount of vandefitemcel further comprises administering the therapeutically effective amount of vandefitemcel to a brain region of the subject.

In some aspects, administering the therapeutically effective amount of vandefitemcel further comprises administering the therapeutically effective amount of vandefitemcel to at least one of an entorhinal cortex, a corpus callosum, a thalamus, a hypothalamus, an internal capsule, and a cerebral cortex of the subject.

In some aspects, administering the therapeutically effective amount of vandefitemcel further comprises administering the therapeutically effective amount of vandefitemcel to a hippocampus of the subject.

In some aspects, administering the therapeutically effective amount of vandefitemcel further comprises injecting the vandefitemcel at multiple sites within the brain of the subject.

In some aspects, administering the therapeutically effective amount of vandefitemcel further comprises administering the therapeutically effective amount of vandefitemcel to a deposition of tau protein aggregates.

In some aspects, administering the therapeutically effective amount of the vandefitemcel further comprises administering the therapeutically effective amount of the vandefitemcel stereotactically through a burr hole in the skull of a subject.

In some aspects, administering the therapeutically effective amount of vandefitemcel further comprises administering the therapeutically effective amount of vandefitemcel by intracerebral implantation.

In some aspects, administering the therapeutically effective amount of vandefitemcel further comprises administering the therapeutically effective amount of vandefitemcel by parenteral administration.

In some aspects, the therapeutically effective amount of vandefitemcel can be suspended in a sterile isotonic crystalloid solution.

In some aspects, the therapeutically effective amount of vandefitemcel can be between about 1.0 million cells and 10.0 million cells (e.g., about 1.0 million cells, 1.5 million cells, 2.0 million cells, 2.5 million cells, 3.0 million cells, 3.5 million cells, 4.0 million cells, 4.5 million cells, 5.0 million cells, 5.5 million cells, 6.0 million cells, 6.5 million cells, 7.0 million cells, 7.5 million cells, 8.0 million cells, 8.5 million cells, 9.0 million cells, 9.5 million cells, 10.0 million cells, and amounts therebetween).

In some aspects, the vandefitemcel can be made by a method comprising: providing a culture of mesenchymal stem cells; contacting the culture of mesenchymal stem cells with a polynucleotide encoding a Notch intracellular domain (NICD), wherein the polynucleotide does not encode a full-length Notch protein, selecting cells that include the polynucleotide; and further culturing the selected cells in the absence of selection for the polynucleotide.

In some aspects, the mesenchymal stem cells can be human bone marrow-derived cells.

In some aspects, a method of reducing tau protein aggregates is disclosed. The method can comprise administering vandefitemcel to a deposition of tau protein aggregates.

In some aspects, administering the vandefitemcel further comprises administering a therapeutically effective amount of vandefitemcel to the deposition of tau protein aggregates within the brain of a subject.

In some aspects, administering the vandefitemcel further comprises administering the therapeutically effective amount of vandefitemcel to an entorhinal cortex, a corpus callosum, a thalamus, a hypothalamus, an internal capsule, and a cerebral cortex of the subject.

In some aspects, administering the vandefitemcel further comprises administering the therapeutically effective amount of vandefitemcel to a hippocampus of the subject.

In some aspects, administering the vandefitemcel further comprises injecting the vandefitemcel at multiple sites within the brain of the subject.

In some aspects, administering the vandefitemcel further comprises administering the therapeutically effective amount of the vandefitemcel stereotactically through a burr hole in the skull of a subject.

In some aspects, administering the vandefitemcel further comprises administering a therapeutically effective amount of vandefitemcel by intracerebral implantation.

In some aspects, administering the vandefitemcel further comprises administering a therapeutically effective amount of vandefitemcel by parenteral administration.

In some aspects, the vandefitemcel can be suspended in a sterile isotonic crystalloid solution.

In some aspects, the vandefitemcel administered can be between about 1.0 million cells and 10.0 million cells (e.g., about 1.0 million cells, 1.5 million cells, 2.0 million cells, 2.5 million cells, 3.0 million cells, 3.5 million cells, 4.0 million cells, 4.5 million cells, 5.0 million cells, 5.5 million cells, 6.0 million cells, 6.5 million cells, 7.0 million cells, 7.5 million cells, 8.0 million cells, 8.5 million cells, 9.0 million cells, 9.5 million cells, 10.0 million cells, and amounts therebetween).

In some aspects, the vandefitemcel can be made by a method comprising: providing a culture of mesenchymal stem cells; contacting the culture of mesenchymal stem cells with a polynucleotide encoding a Notch intracellular domain (NICD), wherein the polynucleotide does not encode a full-length Notch protein, selecting cells that include the polynucleotide; and further culturing the selected cells in the absence of selection for the polynucleotide.

In some aspects, the mesenchymal stem cells are human bone marrow-derived cells.

In some aspects, a composition for treating Alzheimer's disease is disclosed. The composition can comprise vandefitemcel and one or more pharmaceutically acceptable excipients.

In some aspects, the vandefitemcel can be made by a process comprising: providing a culture of mesenchymal stem cells; contacting the culture of mesenchymal stem cells with a polynucleotide encoding a Notch intracellular domain (NICD), wherein the polynucleotide does not encode a full-length Notch protein, selecting cells that include the polynucleotide; and further culturing the selected cells in the absence of selection for the polynucleotide.

In some aspects, the mesenchymal stem cells can be human bone marrow-derived cells.

In some aspects, the mesenchymal stem cells are transiently-transfected with a plasmid comprising the polynucleotide encoding the NICD.

In some aspects, the one or more pharmaceutically acceptable excipients include at least one of buffers, proteins, stabilizers, and preservatives.

In some aspects, the vandefitemcel can be suspended in a sterile isotonic crystalloid solution.

In some aspects, a composition for reducing tau protein aggregates is disclosed. The composition can comprise vandefitemcel; and one or more pharmaceutically acceptable excipients.

In some aspects, the vandefitemcel can be made by a process comprising: providing a culture of mesenchymal stem cells; contacting the culture of mesenchymal stem cells with a polynucleotide encoding a Notch intracellular domain (NICD), wherein the polynucleotide does not encode a full-length Notch protein, selecting cells that include the polynucleotide; and further culturing the selected cells in the absence of selection for the polynucleotide.

In some aspects, the mesenchymal stem cells can be human bone marrow-derived cells.

In some aspects, the mesenchymal stem cells are transiently-transfected with a plasmid comprising the polynucleotide encoding the NICD.

In some aspects, the one or more pharmaceutically acceptable excipients include at least one of buffers, proteins, stabilizers, and preservatives.

In some aspects, the vandefitemcel can be suspended in a sterile isotonic crystalloid solution.

### BRIEF DESCRIPTION OF THE DRAWINGS

FIG. 1 illustrates an example process for generating a tau protein aggregate detection system using three-dimensional (3D) neuronal clusters made from human-induced pluripotent stem cells (hiPSCs).

FIGS. 2A and 2B illustrate that single cell RNA-sequencing and neuron markers were used to reveal that various types of neuronal cells were included in each of the 3D hiPSC-derived neuronal clusters.

FIG. 3 illustrates the progressive inclusion of vandefitemcel into a hiPSC-derived 3D neuronal cell cluster.

FIG. 4 illustrates that different types of fluorescent stains or immunofluorescent stains can be used to visualize vandefitemcel within a neuronal cell cluster.

FIG. 5 are immunofluorescent confocal images showing tau-seeded hiPSC-derived neuronal cell clusters containing vandefitemcel stained using anti-CD44 antibody, anti-GFAP antibody, and MC-1 antibody.

FIG. 6 illustrates that the MC-1 antibody signal was reduced in tau-seeded hiPSC-derived neuronal cell clusters receiving vandefitemcel in a dose-dependent manner.

FIG. 7 illustrates certain subpopulation specific changes in both wild type and mutant neuronal cell clusters with or without recombinant tau seeds and with or without vandefitemcel.

### DETAILED DESCRIPTION

The below terms are defined as follows for purposes of this disclosure.

### Definitions

The terms “administration” and “administering” refer to the physical introduction of a composition comprising a therapeutic agent to a subject, using any of the various methods and delivery systems known to those skilled in the art. For example, routes of administration for vandefitemcel can include intracerebral, intrathecal, or other parenteral routes of administration, for example by injection or infusion. The phrase “parenteral administration” as used herein means modes of administration other than enteral and topical administration, usually by injection and infusion. Administering can also be performed, for example, once, a plurality of times, and/or over one or more extended periods and can be a therapeutically effective dose or a subtherapeutic dose.

“Alzheimer's disease” is a type of progressive brain disease caused by damage to neurons in the brain. Those suffering from Alzheimer's disease usually exhibit an accumulation of beta-amyloid proteins and abnormally phosphorylated tau proteins, as well as the degeneration of neurons. The accumulation of the protein fragment beta-amyloid plaques outside neurons and the accumulation or aggregation of the protein tau (also called tau tangles or neurofibrillary tangles) inside neurons are two of several brain changes associated with Alzheimer's disease. These changes are followed by damage to and destruction of neurons, called neurodegeneration. Beta-amyloid plaque can damage neurons by interfering with neuron-to-neuron communication at synapses. Inside neurons, tau tangles or neurofibrillary tangles block the transportation of nutrients and other molecules essential for the normal function and survival of neurons. Other brain changes associated with Alzheimer's disease include inflammation and brain atrophy (a decrease in brain volume) due to cell loss [1].

The terms “implantation” and “transplantation” are used to denote the introduction of exogenous cells (e.g., vandefitemcel or SB623 cells) into a subject or patient. Exogenous cells can be autologous (i.e. obtained from the subject) or allogeneic (i.e., obtained from an individual other than the subject).

An “isotonic crystalloid solution” is a solution containing water-soluble electrolytes. Such a solution contains the same amount of electrolytes as plasma.

“Mesenchymal cells” refer to cells of mesenchymal tissue (e.g., chondroblasts, chondrocytes, osteoblasts, osteocytes, adipocytes) and their precursors and include, for example, fibroblasts (e.g., human foreskin fibroblasts), MSCs (as defined herein) and cells derived from MSCs such as, for example, vandefitemcel, as defined herein.

“MSCs” (“mesenchymal stem cells”) refer to adherent, non-hematopoietic pluripotent cells obtained from bone marrow. These cells are variously known as mesenchymal stem cells, mesenchymal stromal cells, marrow adherent stromal cells, marrow adherent stem cells and bone marrow stromal cells. MSCs can also be obtained from, e.g., umbilical cord blood, adipose tissue, dental pulp, Wharton's jelly, and various types of connective tissue. MSCs can be obtained by selecting (e.g., by growth in culture) adherent cells (i.e., cells that adhere to tissue culture plastic) from bone marrow. To obtain MSC populations having a sufficient number of cells for use in therapy, populations of adherent cells are expanded in culture after selecting for adherence. Expansion in culture also enriches MSCs, since contaminating cells (such as monocytes) do not proliferate under the culture conditions. Exemplary disclosures of MSCs are provided in U.S. Patent Publication No. 2003/0003090 and the literature [8]. Methods for isolating and purifying MSCs can be found, for example, in U.S. Pat. No. 5,486,359 and the literature [9, 10]. Human MSCs are commercially available (e.g., BioWhittaker, Walkersville, Md.) or can be obtained from donors by, e.g., bone marrow aspiration, followed by culture and selection for adherent bone marrow cells. See, e.g., WO 2005/100552. MSCs can also be isolated from umbilical cord blood [11, 12]. Additional sources of MSCs include, for example, adipose tissue, dental pulp, and Wharton's jelly.

A “neurodegenerative disorder is a type of disease in which cells of the central nervous system stop working or die. Some neurodegenerative disorders are characterized by the presence of tau protein aggregates or neurofibrillary tangles. Such disorders are also referred to as “tauopathies.” Alzheimer's disease is an example of a tauopathy. Other tauopathies include neurofibrillary tangle dementia, aging-related tau astrogliopathy, frontotemporal dementia, and Pick's disease.

The “Notch protein” (e.g., Notch 1 protein) is a transmembrane receptor, found in all metazoans, that influences cell differentiation through intracellular signaling. Contact of the Notch extracellular domain (e.g., the extracellular domain of the Notch 1 protein) with a Notch ligand (e.g., Delta, Serrate, Jagged) results in two proteolytic cleavages of the Notch protein, the second of which is catalyzed by a γ-secretase and releases the Notch intracellular domain (NICD) into the cytoplasm. In the mouse Notch protein, this cleavage occurs between amino acids gly1743 and val1744. The NICD translocates to the nucleus, where it acts as a transcription factor, recruiting additional transcriptional regulatory proteins (e.g., MAM, histone acetylases) to relieve transcriptional repression of various target genes (e.g., Hes 1). Additional details and information regarding Notch signaling can be found in the literature [13, 14, 15].

The term “pharmaceutically acceptable excipient” refers to an excipient for administration of a pharmaceutical agent. These “excipients” refer to any substance other than the active pharmaceutical agent. Examples of pharmaceutically acceptable excipients include, but are not limited to, buffers, proteins, carbohydrates, oleochemicals, petrochemicals, stabilizers, preservatives, fillers, diluents, binders, viscosity agents, coatings, disintegrants, colorants, and lubricants.

“Tau protein aggregates” is an important pathological hallmark or signature in multiple neurodegenerative disorders including Alzheimer's disease. Tau is a neuron specific microtubule-associated protein that regulates microtubule stability. Such proteins are critical for axonal outgrowth and synaptic plasticity. In a pathological or diseased state, tau dissociates from microtubules and forms insoluble aggregates called neurofibrillary tangles (NFTs). The accumulation of NFTs in neurons leads to neuronal degeneration.

The terms “vandefitemcel,” vandefitemcel cells,” “SB623,” and “SB623 cells” refer to populations of cells obtained following transient expression of an exogenous Notch intracellular domain (NICD) in MSCs. For example, a population of vandefitemcel or SB623 cells can be obtained by transient transfection of MSCs with a vector comprising sequences encoding a NICD (e.g., from the human Notch 1 protein) but not encoding a full-length Notch protein followed by selection (e.g., with G418). The selected cells can be further cultured in a standard culture medium, optionally supplemented with a serum, in the absence of any added growth factors or differentiation factors (other than those which may be present in the serum, if serum is present in the culture medium). Vandefitemcel can be derived from human (allogeneic) bone marrow MSCs by transient transfection of human bone marrow MSCs with NICD (e.g., the human Notch1 intracellular domain (NICD1)), followed by selection, and subsequent expansion. This process produces a cell population that is different than the parental MSCs [5, 6, 7]. Vandefitemcel or SB623 cells have also been referred to as descendants of NICD transiently-transfected MSCs (“DNTT-MSCs”).

“Therapeutically effective” means the amount of an agent required to provide a meaningful patient benefit or promote disease regression evidenced by a decrease in severity of disease symptoms, an increase in frequency and duration of disease symptom-free periods, or a prevention of impairment or disability due to the disease affliction. The therapeutically effective amount of an agent can be evaluated using a variety of methods known to the skilled practitioner such as a practitioner in the field of neurology.

### Preparation of Vandefitemcel

The vandefitemcel administered are allogeneic cells descended from mesenchymal stem cells transiently-transfected by a polynucleotide encoding a Notch intracellular domain (NICD). The cells can be made by a method comprising providing a culture of the mesenchymal stem cells, contacting the culture of mesenchymal stem cells with the polynucleotide encoding an NICD (where the polynucleotide does not encode a full-length Notch protein), selecting cells that comprise the polynucleotide, and further culturing the selected cells in the absence of selection for the polynucleotide. The mesenchymal stem cells can be human bone marrow-derived cells.

As previously discussed, vandefitemcel can be obtained from marrow adherent stromal cells, also known as MSCs, by transiently expressing the intracellular domain of the Notch protein in the MSCs. Transient expression of the Notch intracellular domain (e.g., the NICD from the human Notch 1 protein) in an MSC can be sufficient to convert a population of MSCs into a population of vandefitemcel. Additional treatment with growth and/or differentiation factors is not required. Thus, a population of MSCs can be converted to a population of vandefitemcel by transient transfection of MSCs with a vector comprising sequences encoding a NICD (but not encoding full-length Notch protein), followed by selection for cells comprising the vector and further culture of the selected cells in serum-containing medium, in the absence of exposure to additional growth and/or differentiation factors. See, for example, U.S. Pat. Nos. 7,682,825; 8,945,919; and WO 2009/023251; the contents of which are incorporated herein by reference in their entireties for the purposes of describing isolation of mesenchymal stem cells and conversion of mesenchymal stem cells to vandefitemcel (also referred to as “neural precursor cells” and “neural regenerating cells” in those documents).

In this disclosure, any polynucleotide encoding a Notch intracellular domain (e.g., vector) can be used, and any method for the selection and enrichment of transfected cells can be used. For example, MSCs can be transfected with a vector containing sequences encoding a Notch intracellular domain (e.g., the human Notch 1 intracellular domain) and also containing sequences encoding a selection marker (e.g., drug resistance; e.g., resistance to G418). In some instances, two vectors, one containing sequences encoding a Notch intracellular domain and the other containing sequences encoding a drug resistance marker, can be used for transfection of MSCs. In these instances, selection is achieved, after transfection of a cell culture with the vector or vectors, by adding a selective agent (e.g., G418) to the cell culture in an amount sufficient to kill cells that do not comprise the vector but spare cells that do. Absence of selection entails the removal of said selective agent or reduction of its concentration to a level that does not kill cells that do not comprise the vector. Following selection (e.g., for seven days) the selective agent can be removed and the cells can be further cultured (e.g., for two passages) in serum-containing culture medium.

It is also possible, depending on the nature of the selection marker and/or the concentration of the selective agent used, that not every cell that lacks a vector encoding a selection marker will be killed during the selection process. For example, a selective agent may inhibit growth of a cell not comprising the selection marker and, after removal of the selective agent, that cell may recover and resume growth.

Preparation of vandefitemcel thus involves transient expression of an exogenous Notch intracellular domain in an MSC. To this end, MSCs can be transfected with a vector comprising sequences encoding a Notch intracellular domain (e.g., the human Notch 1 intracellular domain) wherein said sequences do not encode a full-length Notch protein. All such sequences are known and readily available to those of skill in the art [14].

Similar information is available for Notch proteins and nucleic acids from additional species, including rat, Xenopus, Drosophila and human [16, 17]. Additional information can also be found in NCBI Reference Sequence No. NM_017167, SwissProt P46531, SwissProt Q01705, and GenBank CAB40733. The foregoing references are incorporated by reference in their entireties for the purpose of disclosing the amino acid sequence of the full-length Notch protein and the amino acid sequence of the Notch intracellular domain in a number of different species.

In some instances, vandefitemcel can be prepared by introducing, into MSCs, a nucleic acid comprising sequences encoding a Notch intracellular domain such that the MSCs do not express exogenous Notch extracellular domain. Such can be accomplished, for example, by transfecting MSCs with a vector comprising sequences encoding a Notch intracellular domain wherein said sequences do not encode a full-length Notch protein.

Additional details on the preparation of vandefitemcel or SB623 cells, and methods for making cells with properties similar to those of vandefitemcel or SB623 cells which can be used in the methods disclosed herein, can be found in the literature and U.S. Pat. Nos. 7,682,825; 8,945,919; and 9,441,199, the contents of which are incorporated herein by reference in their entireties for the purposes of describing alternative methods for the preparation of, vandefitemcel, and for providing methods for making cells with properties similar to those of vandefitemcel.

### Transfection

Methods for introduction of exogenous DNA into cells (i.e., transfection), and selection of transfected cells, are known in the art [19, 20].

Preparation of Tau Protein Aggregate Detection System Using 3D hiPSC-Derived Neuronal Clusters

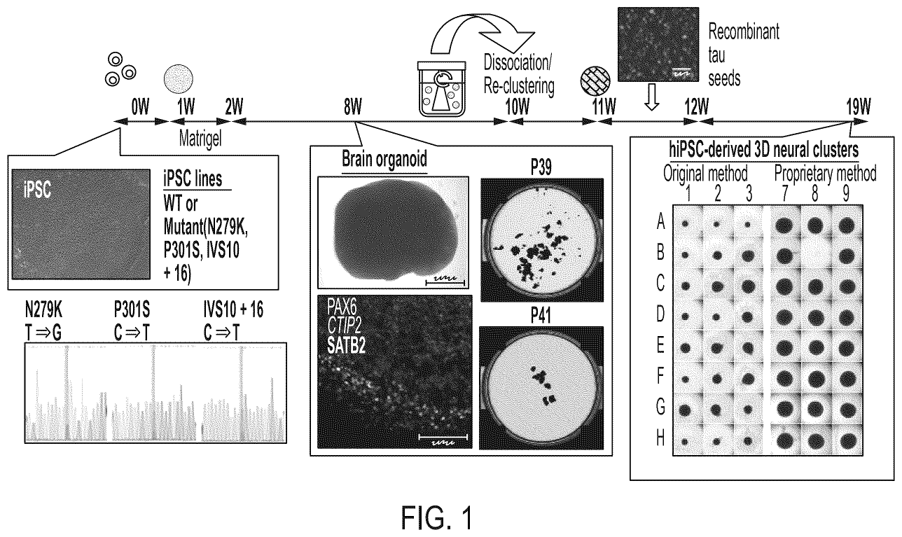

A tau protein aggregate detection system was prepared using human-induced pluripotent stem cells (hiPSCs) established from endothelial progenitor cells obtained from the peripheral blood of a healthy donor and cells in which tau mutations were introduced by genome editing (see FIG. 1 and Example 1). The genome-edited cells had triple mutations in the tau genes N279K, P301S, and IVS10+16. All such cells were cultivated and cultured at high-densities. Cultured cells were then induced to become neuronal cells. After several weeks, the cells formed into brain organoids with a cerebral cortex-like structure. To maintain uniformity, the cells forming the brain organoids were first dispersed or dissociated and then 24 reaggregated or re-clustered between 10 weeks and 11 weeks to form uniform neuronal cell aggregates or neuronal cell clusters. As part of the reaggregation or re-clustering process, recombinant tau seeds were introduced to cause tau protein aggregation in the uniform neuronal cell aggregates. The formation of tau protein aggregates in the neuronal cell clusters was successfully confirmed by immunostaining with MC-1.

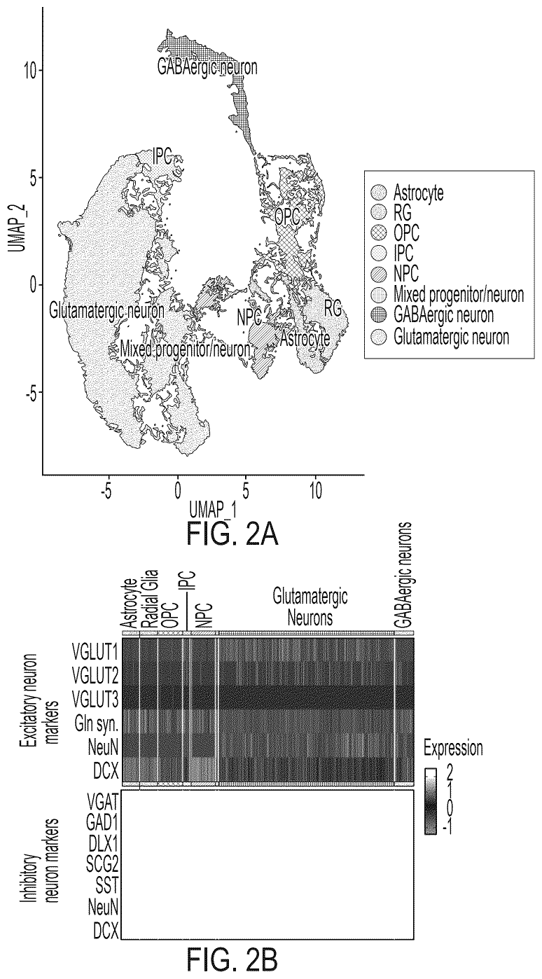

Single cell RNA-sequencing and neuron markers revealed that various types of neuronal cells were included in each of the 3D hiPSC-derived neuronal clusters. These included glutamatergic neurons, GABAergic neurons, neural progenitor cells (NPCs), mixed progenitors and neurons, radial glial (RG) cells, intermediate progenitor cells (IPCs), oligodendrocyte progenitor cells (OPCs), and astrocytes (see FIGS. 2A and 2B). As previously discussed, tau protein aggregates were visualized using MC-1 antibody.

Thus, it was discovered that the brain organoids made using tau-seeded human-induced pluripotent stem cells derived neuronal clusters could be useful for modeling the pathology of neurodegenerative disorders characterized by the presence of tau protein aggregates.

### Effects of Vandefitemcel on Tau Protein Aggregates

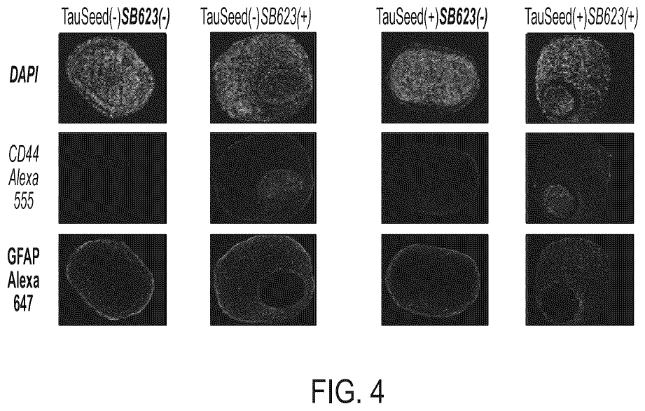

Different amounts of vandefitemcel were introduced to the 3D hiPSC-derived neuronal clusters containing tau protein aggregates. The amount of vandefitemcel ranged from 2×10\hat{ }3 cells per neuronal cell cluster to 1×10\hat{ }4 cells per neuronal cell cluster. Anti-CD44 antibody worked well to visualize vandefitemcel within the neuronal clusters (see FIG. 4).

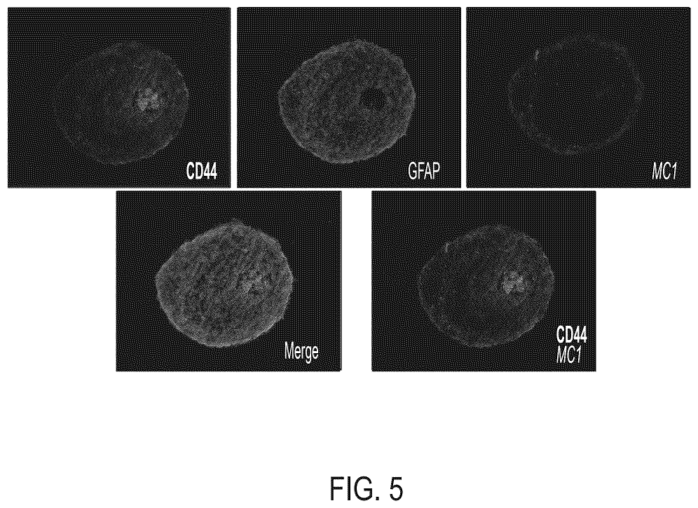

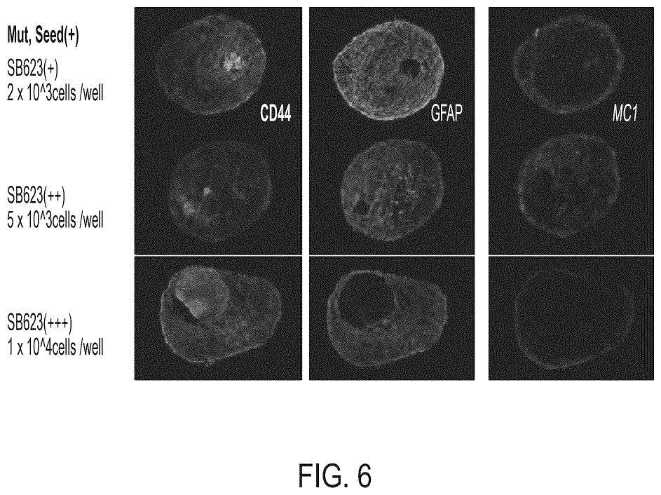

One unexpected discovery made by the applicant is that the MC-1 antibody signal (fluorescence intensity) was attenuated around the vandefitemcel (see FIG. 5). Moreover, the MC-1 antibody signal (fluorescence intensity) was reduced in tau-seeded hiPSC-derived neuronal cell clusters receiving vandefitemcel in a dose-dependent manner (see FIG. 6). Thus, vandefitemcel reduces tau protein aggregates in brain organoids made to mimic neuronal cells afflicted by tau aggregation in vivo. Since brain organoids, such as the hiPSC-derived neuronal clusters disclosed herein, have shown promise as effective preclinical pathological models [21, 22], the discoveries made by the applicant using such brain organoids can likely be applied to human patients suffering from neurodegenerative disorders characterized by the presence of tau protein aggregates.

### Methods of Treatment

One unexpected result from the experiments described herein is that the introduction of vandefitemcel reduces tau protein aggregates in neuronal cell. Since tau protein aggregates, in the form of neurofibrillary tangles (NFTs), are the pathological hallmarks of neurodegenerative disorders such as Alzheimer's disease, this discovery provides a new method for the treatment of neurodegenerative disorders characterized by the presence of tau protein aggregates. More specifically, this discovery provides new a method for the treatment of Alzheimer's disease.

Thus, disclosed is a method of treating Alzheimer's disease, comprising administering a therapeutically effective amount of vandefitemcel to a subject (e.g., a human subject or an animal subject) in need thereof. Administering the therapeutically effective amount of vandefitemcel can further comprise administering the therapeutically effective amount of vandefitemcel to a brain region of the subject (e.g., a brain region characterized by the presence of tau protein aggregates).

Administering the therapeutically effective amount of vandefitemcel can also comprise administering the therapeutically effective amount of vandefitemcel to an entorhinal cortex, a corpus callosum, a thalamus, a hypothalamus, an internal capsule, and a cerebral cortex of the subject.

Administering the therapeutically effective amount of vandefitemcel can further comprise administering the therapeutically effective amount of vandefitemcel to a hippocampus of the subject.

Administering the therapeutically effective amount of vandefitemcel further comprises injecting the vandefitemcel at multiple sites within the brain of the subject. In some cases, the vandefitemcel can be administered stereotactically via a burr hole in the skull of a subject.

Additional details on stereotactic administration of the cells can be found in U.S. Pat. No. 11,439,761, the content of which is incorporated herein by reference in its entirety for the purposes of describing stereotactic administration of vandefitemcel and equipment used for such purposes.

Administering the therapeutically effective amount of vandefitemcel can also comprise administering the therapeutically effective amount of vandefitemcel to a deposition of tau protein aggregates.

Administering the therapeutically effective amount of vandefitemcel can also comprise administering the therapeutically effective amount of vandefitemcel by parenteral administration. For example, the vandefitemcel can be administered by intracerebral implantation.

The therapeutically effective amount of vandefitemcel can be between about 1.0 million cells and 10.0 million cells (e.g., about 1.0 million cells, 1.5 million cells, 2.0 million 5.0 million cells, 5.5 million cells, 6.0 million cells, 6.5 million cells, 7.0 million cells, 7.5 million cells, 8.0 million cells, 8.5 million cells, 9.0 million cells, 9.5 million cells, 10.0 million cells, and amounts therebetween).

The vandefitemcel can be suspended in a pharmaceutically acceptable carrier or diluent. In some instances, the pharmaceutically acceptable carrier or diluent can be a sterile isotonic crystalloid solution. For example, the cell suspension can comprise the cells suspended in Plasma-Lyte™ A (Baxter Healthcare Corporation). The cells can also be suspended in another physiologically compatible carrier such as phosphate buffered saline.

As disclosed herein, the vandefitemcel can be made by a method comprising: (i) providing a culture of mesenchymal stem cells; (ii) contacting the culture of mesenchymal stem cells with a polynucleotide encoding a Notch intracellular domain (NICD) and where the polynucleotide does not encode a full-length Notch protein; (iii) selecting cells that comprise the polynucleotide; and (iv) further culturing the selected cells in the absence of selection for the polynucleotide. The mesenchymal stem cells can be human bone marrow-derived cells.

Also disclosed is a method of treating a neurodegenerative disorder characterized by the presence of tau protein aggregates comprising administering a therapeutically effective amount of vandefitemcel to a subject in need thereof.

Administering the therapeutically effective amount of vandefitemcel can comprise administering the therapeutically effective amount of vandefitemcel to a brain region of the subject. For example, administering the therapeutically effective amount of vandefitemcel can further comprise administering the therapeutically effective amount of vandefitemcel to an entorhinal cortex, a corpus callosum, a thalamus, a hypothalamus, an internal capsule, and a cerebral cortex of the subject.

Administering the therapeutically effective amount of vandefitemcel can further comprise administering the therapeutically effective amount of vandefitemcel to a hippocampus of the subject.

Administering the therapeutically effective amount of vandefitemcel further comprises injecting the vandefitemcel at multiple sites within the brain of the subject. In some cases, the vandefitemcel can be administered stereotactically via a burr hole in the skull of a subject. Administering the therapeutically effective amount of vandefitemcel can also comprise administering the therapeutically effective amount of vandefitemcel to a deposition of tau protein aggregates.

Administering the therapeutically effective amount of vandefitemcel can also comprise administering the therapeutically effective amount of vandefitemcel by parenteral administration. For example, the vandefitemcel can be administered by intracerebral implantation.

The therapeutically effective amount of vandefitemcel can be between about 1.0 million cells and 10.0 million cells (e.g., about 1.0 million cells, 1.5 million cells, 2.0 million cells, 2.5 million cells, 3.0 million cells, 3.5 million cells, 4.0 million cells, 4.5 million cells, 5.0 million cells, 5.5 million cells, 6.0 million cells, 6.5 million cells, 7.0 million cells, 7.5 million cells, 8.0 million cells, 8.5 million cells, 9.0 million cells, 9.5 million cells, 10.0 million cells, and amounts therebetween).

The vandefitemcel can be suspended in a pharmaceutically acceptable carrier or diluent. In some instances, the pharmaceutically acceptable carrier or diluent can be a sterile isotonic crystalloid solution. For example, the cell suspension can comprise the cells suspended in Plasma-Lyte™ A (Baxter Healthcare Corporation). The cells can also be suspended in another physiologically compatible carrier such as phosphate buffered saline.

As disclosed herein, the vandefitemcel can be made by a method comprising: (i) providing a culture of mesenchymal stem cells; (ii) contacting the culture of mesenchymal stem cells with a polynucleotide encoding a Notch intracellular domain (NICD) and where the polynucleotide does not encode a full-length Notch protein; (iii) selecting cells that comprise the polynucleotide; and (iv) further culturing the selected cells in the absence of selection for the polynucleotide. The mesenchymal stem cells can be human bone marrow-derived cells.

Also disclosed is a method of treating a neurodegenerative disorder characterized by the presence of tau protein aggregates comprising administering a therapeutically effective amount of vandefitemcel to a subject in need thereof.

Further disclosed is a method of reducing tau protein aggregates. The method comprising administering vandefitemcel to a deposition of tau protein aggregates. The vandefitemcel can be administered to the deposition of tau protein aggregates within the brain of a subject.

For example, the vandefitemcel can be administered to at least one of an entorhinal cortex, a corpus callosum, a thalamus, a hypothalamus, an internal capsule, a cerebral cortex, and a hippocampus of the subject.

Administering the vandefitemcel can further comprise injecting the vandefitemcel at multiple sites within the brain of the subject. For example, the vandefitemcel can be administered stereotactically via a burr hole in the skull of a subject.

Administering the therapeutically effective amount of vandefitemcel can also comprise administering the therapeutically effective amount of vandefitemcel by parenteral administration. For example, the vandefitemcel can be administered by intracerebral implantation.

The therapeutically effective amount of vandefitemcel can be between about 1.0 million cells and 10.0 million cells (e.g., about 1.0 million cells, 1.5 million cells, 2.0 million cells, 2.5 million cells, 3.0 million cells, 3.5 million cells, 4.0 million cells, 4.5 million cells, 5.0 million cells, 5.5 million cells, 6.0 million cells, 6.5 million cells, 7.0 million cells, 7.5 million cells, 8.0 million cells, 8.5 million cells, 9.0 million cells, 9.5 million cells, 10.0 million cells, and amounts therebetween).

The vandefitemcel can be suspended in a pharmaceutically acceptable carrier or diluent. In some instances, the pharmaceutically acceptable carrier or diluent can be a sterile isotonic crystalloid solution. For example, the cell suspension can comprise the cells suspended in Plasma-Lyte™ A (Baxter Healthcare Corporation). The cells can also be suspended in another physiologically compatible carrier such as phosphate buffered saline.

As disclosed herein, the vandefitemcel can be made by a method comprising: (i) providing a culture of mesenchymal stem cells; (ii) contacting the culture of mesenchymal stem cells with a polynucleotide encoding a Notch intracellular domain (NICD) and where the polynucleotide does not encode a full-length Notch protein; (iii) selecting cells that comprise the polynucleotide; and (iv) further culturing the selected cells in the absence of selection for the polynucleotide. The mesenchymal stem cells can be human bone marrow-derived cells.

The methods can further comprise subjecting a formulated dose of the vandefitemcel to post-release testing prior to administering the cells to the subject.

The therapeutically effective amount of vandefitemcel can be between about 1.0 million cells and 10.0 million cells (e.g., about 1.0 million cells, 1.5 million cells, 2.0 million 5.0 million cells, 5.5 million cells, 6.0 million cells, 6.5 million cells, 7.0 million cells, 7.5 million cells, 8.0 million cells, 8.5 million cells, 9.0 million cells, 9.5 million cells, 10.0 million cells, and amounts therebetween).

### Composition, Formulations, and Kits

Also disclosed are compositions, formulations, and kits for reducing tau protein aggregates and for treating Alzheimer's disease or another neurodegenerative disorder characterized by the presence of tau protein aggregates. The composition can comprise a therapeutically effective amount of cells and a pharmaceutically acceptable carrier or diluent. As previously discussed, the vandefitemcel can be descended from mesenchymal stem cells transiently-transfected by a polynucleotide encoding a Notch intracellular domain (NICD). The vandefitemcel can be made by a process comprising providing a culture of the mesenchymal stem cells (e.g., human bone marrow-derived cells) and contacting the culture of mesenchymal stem cells with the polynucleotide encoding the NICD. For example, the mesenchymal stem cells can be transiently-transfected with a plasmid comprising the polynucleotide encoding the NICD. The process can further comprise selecting cells that comprise the polynucleotide, and further culturing the selected cells in the absence of selection for the polynucleotide. In certain instances, the polynucleotide does not encode a full-length Notch protein. The compositions disclosed herein can be useful for, inter alia, stimulating proliferation and differentiation of neural precursor cells and/or endothelial cells.

A therapeutically effective amount of the composition can comprise (vandefitemcel) cells of an amount suitable for treatment of Alzheimer's disease or another neurodegenerative disorder characterized by the presence of tau protein aggregates. In some instances, the therapeutically effective amount of the composition can comprise between about 1.0 million cells and 10.0 million cells (e.g., about 1.0 million cells, 1.5 million cells, 2.0 million cells, 2.5 million cells, 3.0 million cells, 3.5 million cells, 4.0 million cells, 4.5 million cells, 5.0 million cells, 5.5 million cells, 6.0 million cells, 6.5 million cells, 7.0 million cells, 7.5 million cells, 8.0 million cells, 8.5 million cells, 9.0 million cells, 9.5 million cells, 10.0 million cells, and amounts therebetween).

In other instances, the therapeutically effective amount of the composition can vary based on the nature and severity of the disease or disorder, the weight and general health of the subject and other criteria that are known to those of skill in the art. For example, dosage amounts can vary from about 100; 500; 1,000; 2,500; 5,000; 10,000; 20,000; 50,000; 100,000; 500,000; 1,000,000; 2,500,000; 5,000,000 to 10,000,000 cells or more (or any integral value therebetween); with a frequency of administration of, e.g., a single dose, once per day, twice per week, once per week, twice per month, once per month, depending upon, e.g., body weight, route of administration, severity of disease, etc.

The vandefitemcel can be suspended in a pharmaceutically acceptable carrier or diluent to form the cell suspension. The pharmaceutically acceptable carrier can be a physiologically compatible carrier for implantation. As used herein, the term “physiologically compatible carrier” can refer to a carrier that is compatible with the other ingredients of the formulation and not deleterious to the recipient thereof. Examples of suitable carriers or diluents include cell culture medium (e.g., Eagle's minimal essential medium), phosphate buffered saline, Hank's balanced salt solution+/−glucose (HBSS), and multiple electrolyte solutions. The pharmaceutically acceptable carrier or diluent can also be or comprise a sterile isotonic crystalloid solution such as Plasma-Lyte™ A (Baxter Healthcare Corporation).

The composition can comprise the vandefitemcel packaged in a sealed vial. In some instances, the sealed vial can comprise 0.3 mL of the cell suspension with a cell concentration 19 of approximately 8.5*10⁶ cells/mL. Alternatively, the sealed vial can comprise 0.3 mL of the cell suspension with a cell concentration of approximately 17.0*10⁶ cells/mL.

Also disclosed are other examples of materials which can serve as pharmaceutically-acceptable carriers or excipients including: sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances. Wetting agents, emulsifiers and lubricants, such as sodium lauryl sulfate and magnesium stearate, as well as coloring agents, release agents, coating agents, sweetening, flavoring and perfuming agents, preservatives, and antioxidants can also be present in the composition.

Other examples of pharmaceutically-acceptable carriers or excipients include substances that stimulate angiogenesis (“pro-angiogenic agents”). In some instances, the pro-angiogenic agent can be a protein (e.g., fibroblast growth factor, platelet-derived growth factor, transforming growth factor alpha, hepatocyte growth factor, vascular endothelial growth factor, sonic hedgehog, MAGP-2, HIF-1, PR-39, RTEF-1, c-Myc, TFII, Egr-1, ETS-1) or a nucleic acid encoding such a protein. See, for example, Vincent et al. (2007) Gene Therapy 14:781-789. In other instances, the pro-angiogenic agent can be a small RNA molecule (e.g., siRNA, shRNA, microRNA) or a ribozyme that targets a nucleic acid encoding an inhibitor of angiogenesis. Moreover, the pro-angiogenic agent can be a triplex-forming nucleic acid that binds to DNA sequences regulating the expression of a protein that inhibits angiogenesis, such as to block transcription of the gene encoding the protein.

Exemplary formulations include, but are not limited to, those suitable for parenteral administration, e.g., intrapulmonary, intra-arterial, intra-ocular, intra-cranial, sub-meningeal, or subcutaneous administration, including formulations encapsulated in micelles, liposomes or drug-release capsules (active agents incorporated within a biocompatible coating designed for slow-release); ingestible formulations; formulations for topical use, such as eye drops, creams, ointments and gels; and other formulations such as inhalants, aerosols and sprays. The dosage of the compositions of the disclosure can vary according to the extent and severity of the need for treatment, the activity of the administered composition, the general health of the subject, and other considerations well known to the skilled artisan.

In additional embodiments, the compositions described herein can also be delivered locally. Localized delivery allows for the delivery of the composition non-systemically, thereby reducing the body burden of the composition as compared to systemic delivery. Such local delivery can be achieved, for example, through the use of various medically implanted devices including, but not limited to, stents and catheters, or can be achieved by inhalation, injection or surgery. Methods for coating, implanting, embedding, and otherwise attaching desired agents to medical devices such as stents and catheters are established in the art and contemplated herein.

Another aspect of the present disclosure relates to kits for carrying out the administration of the cells to a subject. For example, the kit can comprise the composition of cells, formulated as appropriate (e.g., in a pharmaceutical carrier), in one or more separate pharmaceutical preparations.

Compositions comprising vandefitemcel can be used in combination with other compositions comprising substances that stimulate angiogenesis (“pro-angiogenic agents”). The compositions can be administered sequentially in any order or concurrently. Accordingly, therapeutic compositions as disclosed herein can contain both vandefitemcel and a pro-angiogenic agent. In additional embodiments, separate therapeutic compositions, one comprising vandefitemcel and the other comprising a pro-angiogenic agent, can be administered to the subject, either separately or together.

The pro-angiogenic agent can be a transcription factor that activates expression of a pro-angiogenic molecule (e.g., protein). Naturally-occurring transcription factors (such as, for example, HIF-1alpha) that regulate the expression of pro-angiogenic proteins, are known. In addition, synthetic transcriptional regulatory proteins can be constructed by genetic engineering. For example, methods for the design of zinc finger DNA-binding domains that bind to a sequence of interest, and methods for the fusion of such zinc finger DNA-binding domains to transcriptional activation and repression domains, have been described. See, for example, U.S. Pat. Nos. 6,534,261:6,607,882; 6,785,613; 6,794,136; 6,824,978; 6,933,1 6,979,539; 7,013,219:7,177,766; 7,220,719; and 7,788,044. These methods can be used to synthesize non-naturally-occurring proteins that activate transcription of any gene encoding a pro-angiogenic protein. hi addition, synthetic zinc finger transcriptional activators of the vascular endothelial growth factor (VEGF) gene have been described. See, e.g., U.S. Pat. Nos. 7,026,462; 7,067,317; 7,560,440:7,605,140; and 8,071,564. Accordingly, a non-naturally-occurring (i.e., synthetic) zinc finger protein that activates transcription of the VEGF gene can be used, in combination with vandefitemcel, for augmenting angiogenesis, e.g., in the treatment of stroke. Furthermore, a natural or synthetic transcriptional regulatory protein (e.g., a synthetic zinc finger transcriptional regulatory protein) that inhibits transcription of an anti-angiogenic molecule can also be used as a pro-angiogenic agent.

Also disclosed is a tau protein aggregate detection system or testing system comprising a plurality of three-dimensional (3D) brain organoids formed from neuronal cell aggregates. The 3D brain organoids formed have a cerebral cortex-like structure. The neuronal cell aggregates are formed from human-induced pluripotent stem cells (hiPSCs) established from endothelial progenitor cells obtained from the peripheral blood of human donors. The neuronal cell aggregates comprise cells in which tau mutations were introduced by genome editing. For example, the genome-edited cells have triple mutations in the tau genes N279K, P301S, and IVS10+16. The neuronal cell aggregates are formed from cells cultured at high-density and induced to become neuronal cells. The brain organoids were formed by dispersing or dissociating the cultured and induced cells that formed into cell clusters after about eight weeks and then reaggregating or re-clustering the cells after about two weeks. The brain organoids further comprise tau protein aggregates formed from recombinant tau seeds introduced during the aforementioned re-clustering or reaggregation process. The brain organoids comprise a variety of neuronal cells including glutamatergic neurons, GABAergic neurons, neural progenitor cells (NPCs), mixed progenitors and neurons, radial glial (RG) cells, intermediate progenitor cells (IPCs), oligodendrocyte progenitor cells (OPCs), and astrocytes.

Further disclosed is a method of forming a tau protein aggregate detection or testing system. The method can comprise culturing human-induced pluripotent stem cells (hiPSCs) at high-density. The hiPSCs are established from endothelial progenitor cells obtained from the peripheral blood of human donors. The hiPSCs comprise cells in which tau mutations were introduced by genome editing. For example, the genome-edited cells have triple mutations in the tau genes N279K, P301S, and IVS10+16. The method can also comprise inducing the cells to become neuronal cells and allowing the cells to cluster and aggregate for about eight weeks. The method can further comprise dispersing or dissociating the cultured and induced cells that formed into cell clusters and then re-aggregating or re-clustering the dispersed or dissociated cell clusters after about two weeks to form a plurality of three-dimensional (3D) brain organoids. The 3D brain organoids formed have a cerebral cortex-like structure. The method can also comprise introducing recombinant tau seeds during the re-clustering or reaggregation step to cause tau protein aggregation in the brain organoids. The brain organoids comprise a variety of neuronal cells including glutamatergic neurons, GABAergic neurons, neural progenitor cells (NPCs), mixed progenitors and neurons, radial glial (RG) cells, intermediate progenitor cells (IPCs), oligodendrocyte progenitor cells (OPCs), and astrocytes. The method can further comprise successfully confirming the formation of tau protein aggregates in the brain organoids by immunostaining (e.g., with MC-1).

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1: Preparation of Tau Protein Aggregate Detection System Using 3D hiPSC-Derived Neuronal Clusters

FIG. 1 illustrates an example process for preparing a tau protein aggregate detection system. The tau protein aggregate detection system was prepared using human-induced pluripotent stem cells (hiPSCs) established from endothelial progenitor cells obtained from the peripheral blood of a healthy donor (RPC771 from REPROCELL Inc.) and cells in which tau mutations were introduced by genome editing. The genome-edited cells had triple mutations in the tau genes N279K, P301S, and IVS10+16 (see far right images in FIG. 1). All such cells were cultivated and cultured at high-densities. Cultured cells were then induced to become 19 neuronal cells. At least three cell type markers (e.g., Pax6, Ctip2, and Satb2) were used to identify different types of neuronal cells in the cell culture. After about eight weeks, the cells formed into brain organoids with a cerebral cortex-like structure (see top-right middle image in FIG. 1). However, since it was difficult to keep the size of the brain organoids uniform and the variations in the size of brain organoids was significant (see images comparing two lots of 24 brain organoids, lot P39 and P41, in the middle of FIG. 1), the cells forming the brain organoids were dispersed or dissociated and reaggregated or re-clustered between 10 weeks and 11 weeks to form uniform cell aggregates or cell clusters. As part of the reaggregation or re-clustering process, recombinant tau seeds (a structure that serves as the core of aggregate of recombinant tau proteins) were introduced to cause tau protein aggregation in the uniform cell aggregates. The formation of tau protein aggregates in the neuronal cell clusters was successfully confirmed at around 19 weeks by staining with MC-1, a monoclonal antibody that is commonly used to detect tau protein aggregates in pathological conformations.

### Example 2: 3D hiPSC-Derived Neuronal Clusters Comprises Various Types of Neuronal Cells

FIGS. 2A and 2B illustrate that single cell RNA-sequencing and neuron markers (both excitatory and inhibitory neuron markers) were used to reveal that various types of neuronal cells were included in each of the 3D hiPSC-derived neuronal clusters. These included glutamatergic neurons, GABAergic neurons, neural progenitor cells (NPCs), mixed progenitors and neurons, radial glial (RG) cells, intermediate progenitor cells (IPCs), oligodendrocyte progenitor cells (OPCs), and astrocytes.

### Example 3: Vandefitemcel Inclusion in 3D hiPSC-Derived Neuronal Clusters

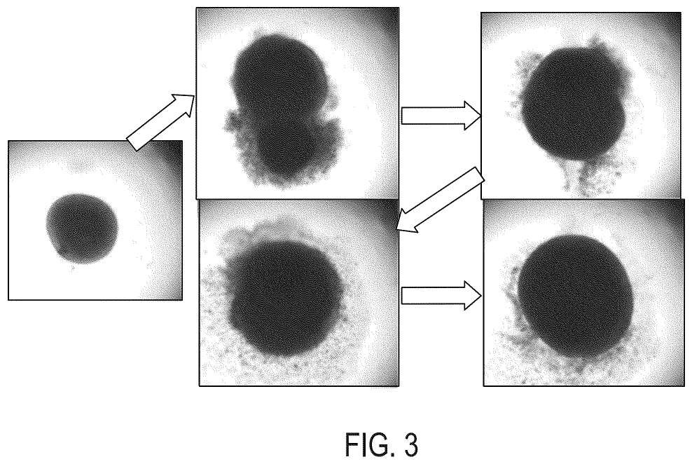

FIG. 3 illustrates the progressive inclusion of vandefitemcel into a hiPSC-derived 3D neuronal cell cluster. The vandefitemcel was introduced during reaggregation.

FIG. 4 illustrates that different types of fluorescent stains or immunofluorescent stains can be used to visualize vandefitemcel (or SB623 cells) within a neuronal cell cluster. As shown in FIG. 4, an anti-CD44 antibody can be used to visualize vandefitemcel (or SB623 cells) and a monoclonal anti-glial fibrillary acidic protein (anti-GFAP) antibody can be used to visualize astrocytes not containing vandefitemcel (or SB623 cells). Cell nuclei were also visualized using 4′,6-diamidino-2-phenylindole (DAPI). As can be seen in FIG. 4, vandefitemcel (or SB623 cells) appear as a small clump or a small cellular mass within the neuronal cell cluster. These stains indicate that vandefitemcel could survive in the neuronal cell clusters.

Non-tau-seeded neuronal clusters (TauSeed(−)) and non-vandefitemcel containing (SB623(−)) neuronal clusters were used as controls. Different amounts of vandefitemcel were introduced ranging from 2×10\hat{ }3 cells (vandefitemcel) per neuronal cell cluster up to 1×10\hat{ }4 cells per neuronal cell cluster. FIG. 4 also illustrates that the anti-CD44 antibody worked well to visualize vandefitemcel within the neuronal clusters in both tau-seeded neuronal clusters and non-tau-seeded neuronal clusters.

### Example 4: The Effects of Vandefitemcel on Tau Protein Aggregates in 3D hiPSC-Derived Neuronal Clusters Using Immunofluorescence Staining

FIG. 5 are immunofluorescent confocal images showing tau-seeded neuronal cell clusters containing vandefitemcel stained using anti-CD44 antibody, anti-GFAP antibody, and MC-1 antibody. The MC-1 antibody was used to visualize tau protein aggregates (e.g., tau fibers and tau oligomers) within the neuronal cell clusters. Also shown are merged images of multiple stains. As can be seen in FIG. 5, the MC-1 antibody signal (fluorescence intensity) was attenuated around the vandefitemcel.

FIG. 6 illustrates that the MC-1 antibody signal (fluorescence intensity) was reduced in tau-seeded hiPSC-derived neuronal cell clusters receiving vandefitemcel in a dose-dependent 8 manner. As shown in FIG. 6, hiPSC-derived 3D neuronal cell clusters were treated with 9 vandefitemcel (SB623) at concentrations of (1) 2×10\hat{ }3 cells per well (or cluster), (2) 5×10\hat{ }3 cells per well (or cluster), and (3) 1×10\hat{ }4 cells per well (or cluster). The neuronal cell clusters were genome-edited (mutant cells) seeded with recombinant tau seeds.

Thus, one unexpected result from the experiments described herein is that the introduction of vandefitemcel reduces tau protein aggregates in 3D hiPSC-derived neuronal cell clusters. Since tau protein aggregates, in the form of neurofibrillary tangles (NFTs), are the pathological hallmarks of neurodegenerative disorders such as Alzheimer's disease, this discovery provides a new method for the treatment of neurodegenerative disorders characterized by the presence of tau protein aggregates. More specifically, this discovery provides new a method for the treatment of Alzheimer's disease.

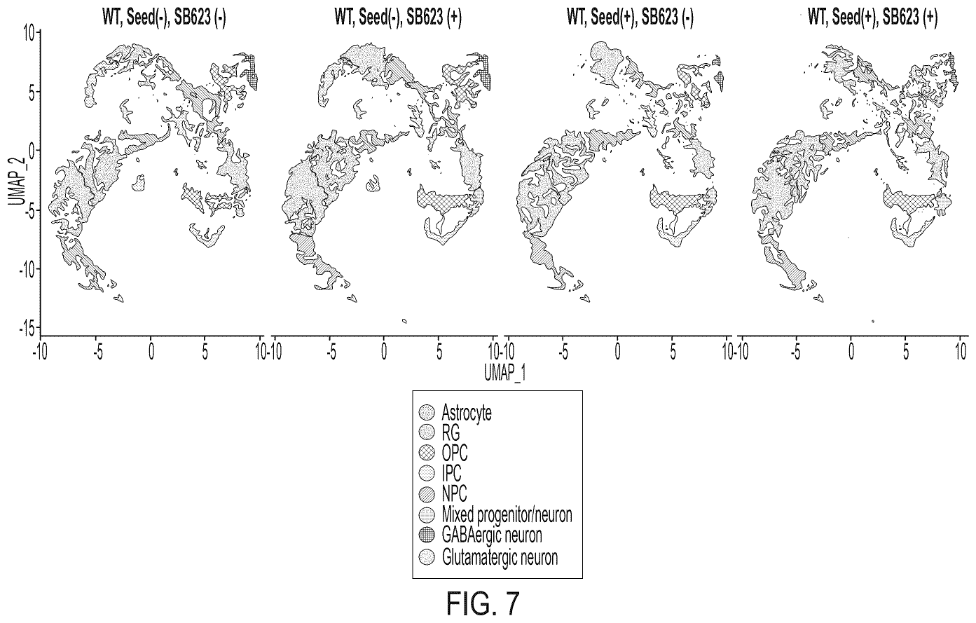

FIG. 7 illustrates certain subpopulation specific changes in both wild type and mutant neuronal cell clusters with or without recombinant tau seeds and with or without vandefitemcel (or SB623 cells). The subpopulations include glutamatergic neurons, GABAergic neurons, neural progenitor cells (NPCs), mixed progenitors and neurons, radial glial (RG) cells, intermediate progenitor cells (IPCs), oligodendrocyte progenitor cells (OPCs), and astrocytes.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned or listed herein (e.g., articles, manuscripts, publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to +0.1%, +1%, +5%, or +10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

### REFERENCES

1. 2023 Alzheimer's disease facts and figures. Alzheimers Dement. 2023; 19 (4): 1598-1695.
2. World Health Organization. Fact sheets of dementia, 15 Mar. 2023, www.who.int/news-room/fact-sheets/detail/dementia.
3. Penney, Jay, William T. Ralvenius, and Li-Huei Tsai. Modeling Alzheimer's disease with iPSC-derived brain cells. Molecular psychiatry 25.1 (2020): 148-167.
4. Kim, C. Kwon, et al. Alzheimer's disease: Key insights from two decades of clinical trial failures. Journal of Alzheimer's Disease 87.1 (2022): 83-100.
5. Aizman I., Tirumalashetty B. J., McGrogan M., Case C. Comparison of the neuropoietic activity of gene-modified versus parental mesenchymal stromal cells and the identification of soluble and extracellular matrix-related neuropoietic mediators. Stem Cell Res Ther. 2014, 5:29.
6. Dao M., Tate C., McGrogan M., Case C. Comparing the angiogenic potency of naïve marrow stromal cells and Notch-transfected marrow stromal cells. J Transl Med. 2013, 11:81.
7. Aizman I., Tate C., McGrogan M., Case C. Extracellular matrix produced by bone marrow stromal cells and by their derivative, SB623 cells, supports neural cell growth. J Neurosci Res. 2009, 87:3198-3206.
8. Jiang, Yuehua, et al. Pluripotency of mesenchymal stem cells derived from adult marrow. Nature 418.6893 (2002): 41-49.
9. Pittenger, Mark F., et al. Multilineage potential of adult human mesenchymal stem cells. Science 284.5411 (1999): 143-147.
10. Dezawa, Mari, et al. Sciatic nerve regeneration in rats induced by transplantation of in vitro differentiated bone-marrow stromal cells. European Journal of Neuroscience 14.11 (2001): 1771-1776.
11. Campagnoli, Cesare, et al. Identification of mesenchymal stem/progenitor cells in human first-trimester fetal blood, liver, and bone marrow. Blood, The Journal of the American Society of Hematology 98.8 (2001): 2396-2402.
12. Erices, Alejandro, Paulette Conget, and José J. Minguell. Mesenchymal progenitor cells in human umbilical cord blood. British journal of haematology 109.1 (2000): 235-242.
13. Artavanis-Tsakonas, Spyros, Kenji Matsuno, and Mark E. Fortini. Notch signaling. Science 268.5208 (1995): 225-232.
14. Mumm, Jeffrey S., and Raphael Kopan. Notch signaling: from the outside in. Developmental biology 228.2 (2000): 151-165.
15. Ehebauer, Matthias, Penelope Hayward, and Alfonso Martinez-Arias. Notch signaling pathway. Science's STKE 2006.364 (2006): cm7-cm7.
16. Weinmaster, Gerry, Veronica J. Roberts, and Greg Lemke. A homolog of Drosophila Notch expressed during mammalian development. Development 113.1 (1991): 199-205.
17. Schroeter, Eric H., Jeffrey A. Kisslinger, and Raphael Kopan. Notch-1 signalling requires ligand-induced proteolytic release of intracellular domain. Nature 393.6683 (1998): 382-386.
18. Dezawa, Mari, et al. Specific induction of neuronal cells from bone marrow stromal cells and application for autologous transplantation. The Journal of clinical investigation 113.12 (2004): 1701-1710.
19. Sambrook, Joseph, Edward F. Fritsch, and Tom Maniatis. Molecular cloning: a laboratory manual. No. Ed. 2. Cold spring harbor laboratory press, 1989.
20. Brent, R., et al. Current Protocols in Molecular Biology (Janssen, K., ed) Vol. 1. (1994).
21. Tang, Xiao-Yan, et al. Human organoids in basic research and clinical applications. Signal transduction and targeted therapy 7.1 (2022): 168.
22. Yang, Siqi, et al. Organoids: The current status and biomedical applications. MedComm 4.3 (2023): e274.

We claim:

1. A method of reducing tau protein aggregates, the method comprising:
administering vandefitemcel to a deposition of tau protein aggregates.

2. The method of claim 1, wherein administering the vandefitemcel further comprises administering a therapeutically effective amount of vandefitemcel to the deposition of tau protein aggregates within the brain of a subject.

3. The method of claim 2, wherein administering the vandefitemcel further comprises injecting the vandefitemcel at multiple sites within the brain of the subject.

4. The method of claim 1, wherein administering the vandefitemcel further comprises administering a therapeutically effective amount of vandefitemcel by intracerebral implantation.

5. The method of claim 1, wherein the vandefitemcel administered is between about 1.0 million cells and 10.0 million cells.

6. The method of claim 1, wherein the vandefitemcel is made by a method comprising:
providing a culture of mesenchymal stem cells, wherein the mesenchymal stem cells are human bone marrow-derived cells;
contacting the culture of mesenchymal stem cells with a polynucleotide encoding a Notch intracellular domain (NICD), wherein the polynucleotide does not encode a full-length Notch protein, selecting cells that comprise the polynucleotide; and
further culturing the selected cells in the absence of selection for the polynucleotide.

7. A method of treating Alzheimer's disease by reducing tau protein aggregates, comprising:
administering a therapeutically effective amount of vandefitemcel to a subject in need thereof.

8. The method of claim 7, wherein administering the therapeutically effective amount of vandefitemcel further comprises administering the therapeutically effective amount of vandefitemcel to a brain region of the subject.

9. The method of claim 7, wherein administering the therapeutically effective amount of vandefitemcel further comprises injecting the vandefitemcel at multiple sites within the brain of the subject.

10. The method of claim 7, wherein administering the therapeutically effective amount of vandefitemcel further comprises administering the therapeutically effective amount of vandefitemcel to a deposition of the tau protein aggregates.

11. The method of claim 7, wherein administering the therapeutically effective amount of vandefitemcel further comprises administering the therapeutically effective amount of vandefitemcel by intracerebral implantation.

12. The method of claim 7, wherein the therapeutically effective amount of vandefitemcel is between about 1.0 million cells and 10.0 million cells.

13. The method of claim 7, wherein the vandefitemcel is made by a method comprising:
providing a culture of mesenchymal stem cells, wherein the mesenchymal stem cells are human bone marrow-derived cells;
contacting the culture of mesenchymal stem cells with a polynucleotide encoding a Notch intracellular domain (NICD), wherein the polynucleotide does not encode a full-length Notch protein, selecting cells that comprise the polynucleotide; and
further culturing the selected cells in the absence of selection for the polynucleotide.

14. A method of treating a neurodegenerative disorder characterized by the presence of tau protein aggregates, the method comprising:
administering a therapeutically effective amount of vandefitemcel to a subject in need thereof.

15. The method of claim 14, wherein administering the therapeutically effective amount of vandefitemcel further comprises administering the therapeutically effective amount of vandefitemcel to a brain region of the subject.

16. The method of claim 15, wherein administering the therapeutically effective amount of vandefitemcel further comprises injecting the vandefitemcel at multiple sites within the brain of the subject.

17. The method of claim 14, wherein administering the therapeutically effective amount of vandefitemcel further comprises administering the therapeutically effective amount of vandefitemcel to a deposition of tau protein aggregates.

18. The method of claim 14, wherein administering the therapeutically effective amount of vandefitemcel further comprises administering the therapeutically effective amount of vandefitemcel by intracerebral implantation.

19. The method of claim 14, wherein the therapeutically effective amount of vandefitemcel is between about 1.0 million cells and 10.0 million cells.

20. The method of claim 14, wherein the vandefitemcel is made by a method comprising:
providing a culture of mesenchymal stem cells, wherein the mesenchymal stem cells are human bone marrow-derived cells;
contacting the culture of mesenchymal stem cells with a polynucleotide encoding a Notch intracellular domain (NICD), wherein the polynucleotide does not encode a full-length Notch protein, selecting cells that comprise the polynucleotide; and
further culturing the selected cells in the absence of selection for the polynucleotide.
